(19) United States
(12) Patent Application Publication
(10) Pub. No.: US 2013/0030549 A1
(43) Pub. Date: Jan. 31, 2013

(54) Leg Prosthesis

(71) Applicants: Mir Saeed Zahedi, London (GB); Graham Harris, Hampshire (GB); Joseph McCarthy, Hampshire (GB)

(72) Inventors: Mir Saeed Zahedi, London (GB); Graham Harris, Hampshire (GB); Joseph McCarthy, Hampshire (GB)

(73) Assignee: BLATCHFORD PRODUCTS LIMITED, Hampshire (GB)

(21) Appl. No.: 13/560,382

(22) Filed: Jul. 27, 2012

(30) Foreign Application Priority Data
Jul. 29, 2011 (GB) 1113208.1

(51) Int. Cl. A61F 2/60; A61F 2/64; A61F 2/62

(52) U.S. Cl. 623/39

(57) ABSTRACT

A lower limb prosthesis for a transfemoral amputee, having a shin portion, ankle portion and a forefoot part of the foot portion formed as an integral fibre-reinforced plastics leaf spring component. This integral component is adjustably mounted to a knee joint which has a knee chassis, a cradle pivotally connected to and depending from the knee chassis, and a flexion control unit in the form of a piston and cylinder assembly interconnecting the knee chassis and the cradle. The shin portion has an elongate resilient section of constant cross-section oriented substantially vertically. The connection of this resilient section to the knee joint cradle and the arrangement of the cradle is such that the vertical distance between the knee axis of rotation and the location of the connection is less than 25% of the vertical distance between the knee axis and the lowest weight-bearing surface of the foot portion.

### FIELD OF THE INVENTION

This invention relates to a lower limb prosthesis and, in particular, to a lower limb prosthesis for a transfemoral amputee.

### BACKGROUND OF THE INVENTION

A known lower limb prosthesis includes a distal limb portion and a knee portion, the knee portion comprising a knee chassis having knee controls; the distal limb portion is then connected to a frame which receives the knee chassis of the knee portion. The knee controls located within the knee chassis control the movement of the distal limb portion. An example of such an arrangement is disclosed in GB 2 432 317.

However, such an arrangement suffers from the disadvantage that the interface between the distal limb portion and the knee chassis does not accommodate a shin portion which can store and return energy during a stride. Furthermore, such known arrangements suffer from the disadvantage that the adaptation of each prosthesis according to the amputee's height is an expensive and material-intensive process.

Lower limb prostheses for below-knee amputees are also known and prostheses of this type include, for example, the arrangement illustrated in U.S. Pat. No. 4,547,913 which includes a single integral member comprising a shin and foot portion and a separate heel portion connected to the foot and shin portion. By providing the single foot and shin portion as an integral resilient member it has been found that this resilience can be used to simulate the energy absorbing and return characteristics of a natural limb. However, such arrangements have not been used for above-knee amputees as the resilience of this member has been found to interfere with the control of the knee portion necessary for control of the limb.

### SUMMARY OF THE INVENTION

Aspects of the invention are set out in the accompanying independent claims. Further aspects of embodiments of the invention are set out in the accompanying dependent claims.

An aspect of the invention provides a lower limb prosthesis comprising a mounting portion, a shin portion, an ankle portion, a foot portion, and a knee joint interconnecting the mounting portion and the shin portion and defining a knee axis of rotation, the knee joint further comprising a flexion control device for controlling rotation of the shin portion relative to the mounting portion, wherein the shin portion comprises an elongate resilient member which is connected to the flexion control device at a location such that, when the prosthesis is in a static standing position, the vertical distance between the knee axis and the location of the said connection is less than 25 percent of the vertical distance between the knee axis and the lowest weight-bearing surface of the foot portion.

The ankle portion and the shin portion may form part of a single integrated member.

At least part of the foot portion, the ankle portion and the shin portion may form part of a single integrated member.

The single integrated member may be composed of a resilient material such as laminated composite carbon fibre.

The shin portion may have a constant cross-section over a predetermined length and may be substantially vertical when the prosthetic is situated at a rest position. The shin portion may be +/−15 degrees to the vertical or may be +/−7 degrees to the vertical.

A further aspect of the invention provides a method of adapting a lower limb prosthesis, the lower limb prosthesis comprising a foot portion, an ankle portion, a resilient shin portion and a knee joint, the prosthesis further comprising a mounting for attaching the knee joint to a residual limb of an amputee and a control unit connected to the mounting and to the shin portion, the control unit regulating movement of the shin portion relative to the mounting, wherein the shin portion is attached to the control unit in a region near to the knee axis, wherein the shin portion extends between the control unit and the ankle portion, and wherein the method comprises:

adapting a length of the shin portion to the height of an amputee; and attaching the control unit to the shin portion at a region proximate to the knee axis.

Embodiments of the invention are hereinafter described by way of example only with reference to the diagrams which are not to scale.

### BRIEF DESCRIPTION OF THE DRAWINGS

In the drawings:

FIG. 1 is a perspective view of a first lower limb prosthesis in accordance with the invention;

FIG. 2 is a lateral view of the first prosthesis;

FIG. 3 is a cross-section of part of the first prosthesis, taken in a sagittal plane and viewed from the medial side;

FIG. 4 is a medial-lateral cross-section on the line B-B in FIG. 3;

FIG. 5 is a perspective view of a second prosthesis in accordance with the invention;

FIG. 6 is another perspective view of the second prosthesis, viewed from the rear;

FIG. 7 is a perspective view of a third prosthesis in accordance with the invention, viewed from above; and

FIG. 8 is a perspective view of a knee portion of the third prosthesis, viewed from above and one side.

### DESCRIPTION OF EXAMPLE EMBODIMENTS

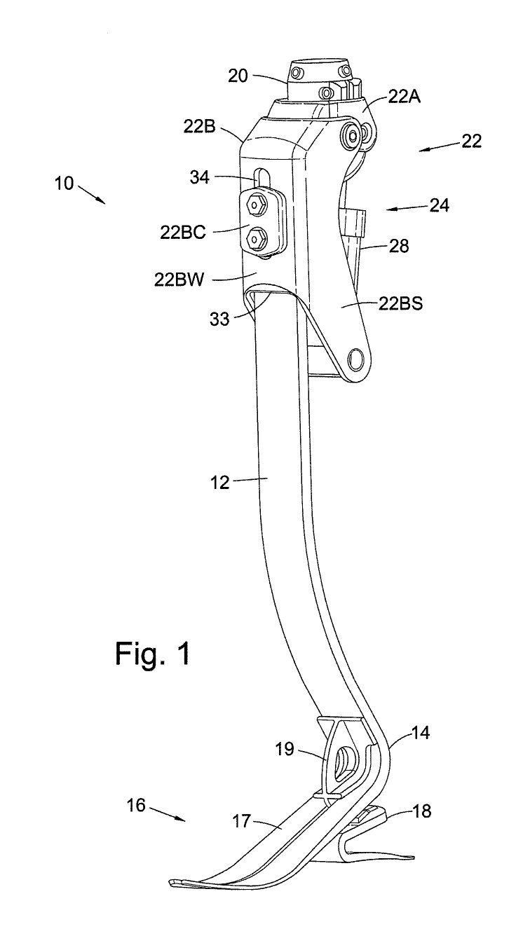

Referring to FIGS. 1 to 4, a first prosthesis 10 according to an embodiment of the invention comprises a shin portion 12, an ankle portion 14 and a foot portion 16. In this embodiment, the shin portion 12, ankle portion 14, and a forefoot part 17 of the foot portion 16 are formed as a single resilient integral member of laminated composite carbon fibre. The use of this material for this application is known and provides energy storage and transmission characteristics which simulate a natural limb. Other fibre-reinforced materials may be used such as materials made with Kevlar®. Although the shin portion 12, ankle portion 14 and the forefoot part 17 are formed as a single, resilient integral member they are referred to herein as separate portions of the same member.

The single, integral member forming the shin portion 12, ankle portion 14 and the forefoot part 17 is a blade. The medial-lateral extent of a cross-section of the blade is significantly greater than the anterior-posterior extent so that the blade forms a leaf spring which can deflect in the anterior-posterior direction when loaded.

The part of the integral member from which the shin portion is formed has a constant cross-section over its length. Although such a constant cross-section is relatively easy to provide where this member is integral, it will be realised that a shin portion having a constant cross-section along its length may also be provided when the shin portion, ankle portion and forefoot part are provided as two or more distinct members.

The foot portion 16 further comprises a heel part 18 connected to the forefoot part 17. The heel part 18 is separate from the integral member forming the forefoot part 17, ankle portion 14 and shin portion 12, but is connected to the forefoot part 17 by a rigid connector 19 in a known manner.

Referring to FIGS. 1 to 3, the prosthesis 10 further includes a mounting portion 20 for mounting the prosthesis to the residual limb of an amputee in a known manner and a knee joint 22 pivotally coupling the mounting portion 20 to the shin portion 12. The mounting portion 20 is a female pyramid receptacle allowing alignment of the prosthesis relative to the residual limb, which is typically received in a socket (not shown). The method and manner in which the shin portion 12 is attached to the mounting portion 20 by the knee joint 22 will now be described in greater detail.

In this embodiment, the knee joint 22 comprises a knee chassis 22A secured to the mounting portion 20, a bracket 22B in the form of a cradle pivotally attached to the knee chassis 22A and a knee flexion control unit 24 which, in this embodiment of the invention, is in the form of a piston and cylinder unit pivotally attached to the knee chassis 22A by upper pivot connections 25 at one end and to the cradle 22B by lower pivot connections 26 at its other end. When a load is applied to the mounting portion 20 of the prosthesis the control unit 24 regulates knee flexion.

As illustrated in FIGS. 1 to 3, the control unit 24 includes a piston rod 27 attached to a piston (not shown) which reciprocates within a cylinder 28. The control unit 24 further comprises a control and actuation mechanism which controls the motion of the piston relative to the cylinder 28 in a known manner. The control and actuation mechanism is known in the art and will therefore not be further described herein. In certain embodiments, cylinder units sold by Chas A Blatchford & Sons Limited under the trade name “Rex II High Activity cylinders” are used.

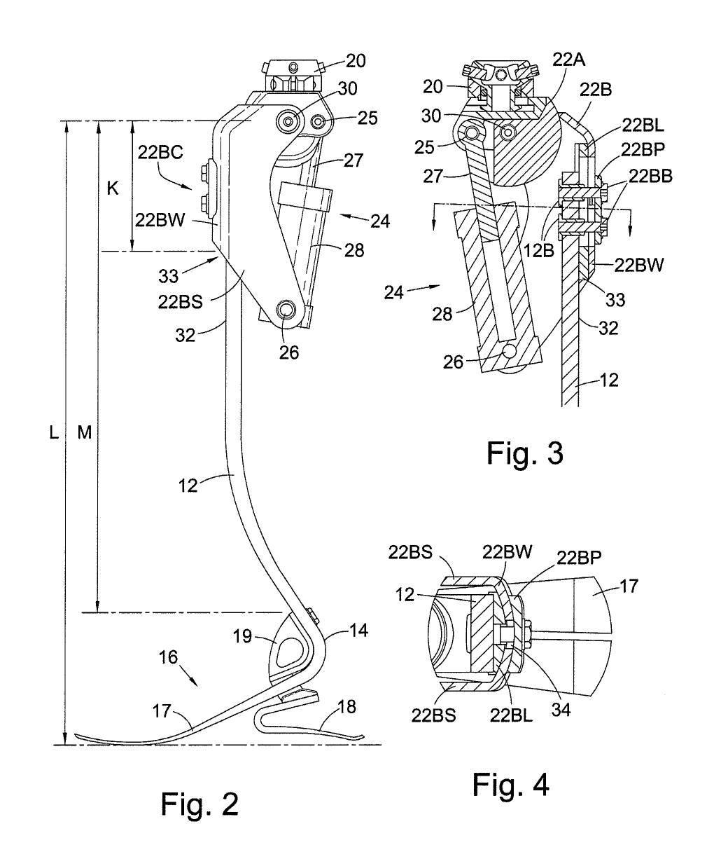

The cradle 22B depends distally from its pivotal connection 30 to the knee chassis 22A and, in a transverse cross-section as shown in FIG. 4, is U-shaped to provide a medial-lateral mounting wall 22BW and side webs 22BS. The side webs 22BS extend distally to the pivotal connections 26 with the lower end of the control unit 24. Together, the medial-lateral wall 22BW of the cradle and the side webs 22BS form a receptacle for the shin portion 12. The medial-lateral wall 22BW includes a lining element 22BL (see FIGS. 3 and 4) to provide a flat posteriorly directed mounting surface for the flat anterior face of the shin portion 12, the latter being secured to the medial-lateral wall 22BW by a clamp 22BC (FIG. 2).

Since the shin portion 12 is of constant cross-section in the superior-inferior direction (in this case a flat blade) and has a major surface 32 which matches the inner surface of the medial-lateral cradle wall 22BW over at least a portion of its length, it may be cut to different lengths to suit the height and gait of the user. The clamp 22BC comprises a pair of bolts 22BB which pass through an anterior clamping plate 22BP and are threaded in bushes 12B housed in the shin portion 12, as shown in FIG. 3. A superior-inferior slot 34 (FIGS. 1 and 4) is formed in the medial-lateral cradle wall 22BW to receive the clamping bolts 22BB and is of sufficient length to allow superior-inferior adjustment of the shin portion 12 with respect to the cradle 22B. Accordingly, the length of the prosthesis is adaptable to the respective amputee not only by virtue of the shin portion 12 having a constant cross-section allowing it to be cut to a suitable length, but also by virtue of it being adjustably secured to the cradle 22B once it has been mounted in the latter.

As best seen in FIGS. 1 and 3, an upper part only of the shin portion 12 engages the inner surface of the medial-lateral cradle wall 22BW so that below this part the shin portion 12 is free to deflect under load. Indeed, the resilient, deflectable part of the shin portion 12 has an upper boundary defined by a lower edge 33 of the medial-lateral cradle wall 22BW. This boundary defines the upper most extent of weight-responsive resilient flexure of the shin portion 12 imposed by its connection via the cradle 22B to the control unit 24. This is the effective height of the connection of the shin portion 12 to the control unit 24 in terms of the position at which a bending moment is applied to the shin portion 12 by the control unit 24 when it resists knee flexion. In summary, therefore the shin portion 12 is resiliently deflectable throughout at least a section of its length, which section terminates proximally at the location of the connection of the shin portion to the control unit.

Since the side webs 22BS extend inferiorly beyond the lower boundary of the connection of the shin portion 12 to the cradle 22B, i.e. below the lower edge 33 of the medial-lateral cradle wall 22BW, the resiliently deflectable part of the shin portion 12 is significantly longer than would be the case if the shin portion 12 were to be connected to the control unit 24 adjacent the lower end of the latter. This increases the capacity of the shin portion 12 for storing energy when it is deflected.

The above-described configuration of the cradle 22B and the connection of the control unit 24 to the shin portion 12 helps to minimise the distance between the region where the control unit 24 connects to the shin portion 12 and the mounting where the prosthesis is connected to the residual limb of the amputee when compared to the dimensions of the shin portion. More particularly, the distance K between the knee axis of rotation defined by the pivotal connection 30, and the connection between the control unit 24 and the shin portion 12, defined by the lower edge 33 of the medial-lateral cradle wall 22BW, is less than 25% of the distance L between the knee axis and the lowest weight-bearing surface of the foot portion when the prosthesis is upright, as shown in FIG. 2. In some embodiments, this percentage may be less than 15%.

In this embodiment of the invention, the lower end of the shin portion 12 is defined by the uppermost extent of the ankle portion 14, i.e. the upper edge of the connector 19 attaching the heel part of the foot portion 16 to the integral member forming the forefoot part 17 of the foot portion 16, the ankle portion 14, and the shin portion 12. The distance K between the knee axis and the connection between the control unit 24 and the shin portion 12 is typically less than 35% of the distance M between the knee axis and the lower end of the shin portion 12, as shown in FIG. 2. In some embodiments, this percentage may be less than 25%. All of the above distances are vertical distances measured when the prosthesis is in a static, upright standing position. In this case, the reference to a “knee axis” applies to a prosthesis with a uniaxial knee joint defined by the pivotal connection 30. In a prosthesis in which the knee joint is in the form of a four-bar linkage with a moving instantaneous axis of rotation, the “knee axis” is to be regarded in the present context as the upper anterior pivot axis of the linkage. By using a greater extent of the shin portion of the prosthesis to contribute to the resilience of the prosthesis than known arrangements, embodiments of the invention which are composed of carbon composite material can be significantly lighter and more durable than other prostheses.

Furthermore, the incorporation of the control unit in embodiments of the invention provides a degree of flexure in the design which, in turn, provides a degree of resilient knee joint flexion from the shin which makes the knee control more predictable due to enhanced proprioceptive feedback in activating, releasing and retaining stance control as this locks or substantially prevents knee joint flexion under load.

By providing a shin portion which has a constant cross-section, and which is vertical when the user is at rest, embodiments of the invention provide a prosthesis which is easy to adapt to a particular user and which can therefore be manufactured and fitted more quickly and cheaply than comparable prior art prostheses could be. In the prosthesis described above with reference to FIGS. 1 to 4, the superior end of the shin portion 12 is enclosed within the cradle 22B. In a variant (not shown), the cradle may be open above its connection to the shin portion 12 so that a greater length of the shin portion 12 may be accommodated above its connection with the cradle 22B.

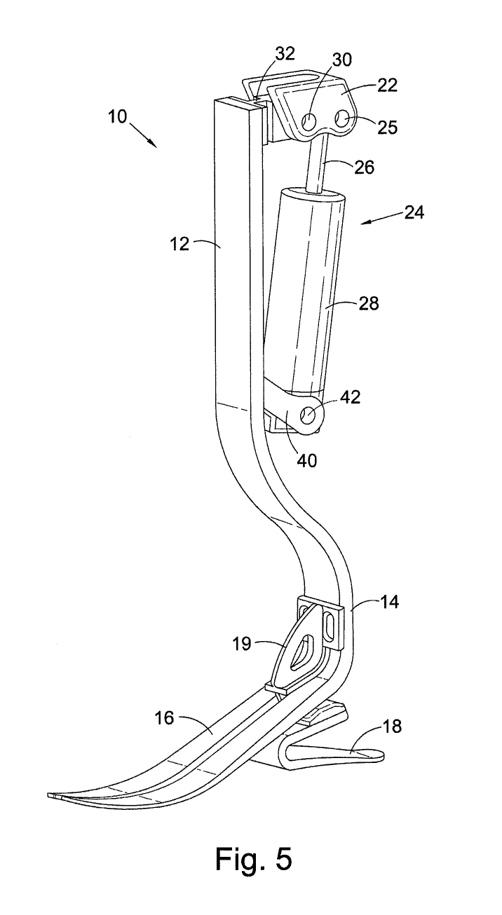

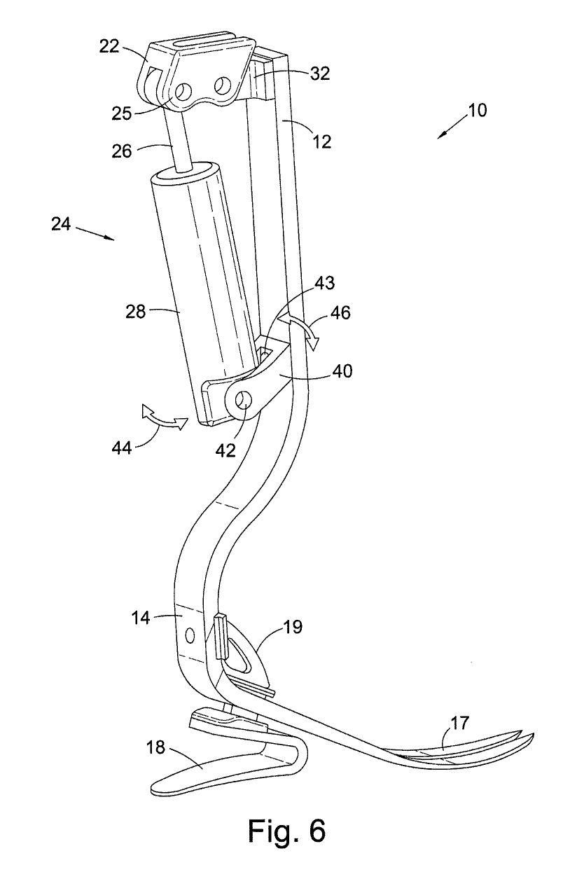

In another prosthesis in accordance with the invention, illustrated in FIGS. 5 and 6, the barrel 28 of the control unit 24 is attached to the shin portion 12 by means of a bracket 40. The bracket 40 is connected to the cylinder by a pin 42 which allows the cylinder to pivot relative to the bracket 40. The bracket 40 is connected to the shin portion 12 by a further pin 43. Referring to FIG. 6, the pin 42 allows the control unit 24 to rotate relative to the shin portion 12 in the directions depicted by arrow 44. The further pin 43 allows the bracket 40 (and thereby the control unit 24) to rotate relative to the shin portion in the direction depicted by arrow 46.

By allowing movement of the cylinder unit 24 relative to the shin portion 12 in the direction of arrows 44 and 46 in the manner described, the knee flexion control provided by the control unit 24 does not result in forces being applied to the shin in a manner or direction which is undesirable and which would diminish or reverse the effect of the cylinder unit 24 on the gait of the user.

In a further embodiment (not shown), an attachment between the control unit and the shin portion is provided which allows rotational movement of the two relative to one another in three rotational directions. This may, for example, be provided by a universal joint or a spherical bearing.

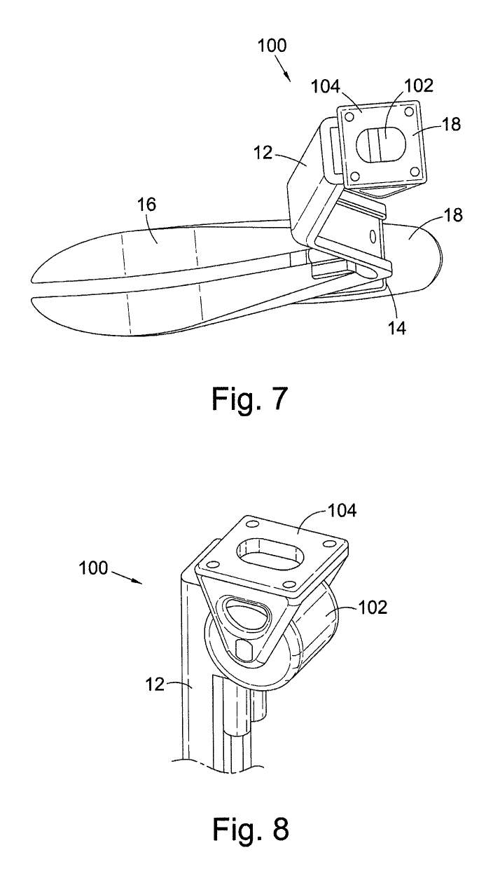

Referring to FIGS. 7 and 8, yet a further lower limb prosthesis 100 in accordance with the invention includes a shin portion 12, ankle portion 14, foot portion 16 and heel portion 18 in the same manner as prosthesis 10 of FIGS. 1 to 4. However, the prosthesis 100 of FIGS. 7 and 8 includes a rotary control unit 102 connected to the shin portion 102 to act as a control unit controlling flexion and extension of the knee joint. A mounting 104 is connected to the rotary control unit 102 and therefore, the shin portion 12 is connected to the rotary control unit 102 in the region of the mounting 104.

The rotary control unit 102 acts in a similar manner to the linear piston and cylinder control unit 24 of the embodiments described above. The rotary control unit 102 provides control of the movement of the mounting 104 relative to the shin portion 12 thereby controlling the flexion of the prosthesis 100 relative to the upper limb of the user to which the prosthetic has been attached. Such rotary control units are well known in the art and may, for example, be the Otto Bock 3R80 knee.

With reference to both the linear and rotary control units, it is known to provide these control units as purely mechanical units which are activated in accordance with position or the load which is brought to bear by the user on the mounting portion of the prosthesis. Alternatively, it is also known to provide such control units with sensors which determine a load, position and/or acceleration of the prosthesis. Therefore, the control provided by these units may be adjusted in accordance with the outputs of these sensors. The control alters the degree to which the control unit allows the corresponding prosthesis to flex (or, in the case of embodiments of this invention, the ease with which the shin portion of the prosthesis moves relative to the mounting).

Since the shin portion is connected to the control unit in the region which is proximate to a mounting portion where the prosthesis is attached to the residual limb of a user, this allows a significant part of the shin portion to contribute to the resilience (energy storage abilities) of the prosthesis. This, in turn, provides a dynamic prosthesis which more closely resembles the action of a natural limb. The prosthesis of embodiments of the invention comprise a control unit acting between the mounting portion and the shin portion. Therefore, having a larger portion of the shin portion contributing to the resilience of the prosthesis allows the control unit to exert a greater degree of control over the prosthesis.

In each of the embodiments of the invention described above with reference to the drawings, the shin portion 12 constitutes a leaf spring of constant cross-section.

The stiffness of the shin portion (and of the other portions of the prosthesis) may be controlled by varying the cross-section of the member from which this is constructed. For example, the cross-section may be provided as a U-channel or as an H-shaped cross-section. Alternatively, features may be formed (by drilling, for example) into the member. Thereby, the axial rotational stiffness as well as axial torque absorption of the device may be controlled. This reduces the discomforting shock loads in axial and rotational directions reaching the stump interface and makes the knee control more predictable due to enhanced proprioceptive feedback in activating, releasing and retaining stance control.

1. A lower limb prosthesis comprising a mounting portion, a shin portion, an ankle portion, a foot portion, and a knee joint interconnecting the mounting portion and the shin portion and defining a knee axis of rotation, the knee joint further comprising a flexion control device for controlling rotation of the shin portion relative to the mounting portion, wherein the shin portion comprises an elongate resilient member which is connected to the flexion control device at a location such that, when the prosthesis is in a static standing position, the vertical distance between the knee axis and the location of the said connection is less than 25 percent of vertical distance between the knee axis and the lowest weight-bearing surface of the foot portion.

2. A prosthesis according to claim 1, wherein the shin portion comprises an elongate resilient section extending distally from the location of said connection to the flexion control device and having a constant cross-section with a major axis oriented in a medial-lateral direction.

3. A prosthesis according to claim 2, wherein said resilient section is oriented substantially vertically when the prosthesis is in a static standing position.

4. A prosthesis according to claim 1, wherein the knee joint comprises an upper knee joint component and a lower knee joint component which is pivotable relative to the upper knee joint component as the knee joint flexes and which has a shin attachment surface to which the shin portion is secured, the attachment surface being substantially vertical when the prosthesis is in a static standing position.

5. A prosthesis according to claim 4, wherein the attachment surface is to the anterior of the said knee axis.

6. A prosthesis according to claim 4, wherein the shin portion is adjustably secured to the lower knee joint component.

7. A prosthesis according to claim 1, wherein the knee joint comprises an upper knee joint component and a lower knee joint component which is pivotable relative to the upper knee joint component as the knee joint flexes, the shin portion being secured to the lower knee joint component, and wherein the flexion control device comprises a variable length strut in the form of a piston and cylinder unit linking the upper and lower knee joint components, the upper knee joint component incorporating an upper pivot and the lower knee joint component incorporating a lower pivot, which pivots serve to connect the piston and cylinder unit to the upper and lower knee joint components respectively.

8. A prosthesis according to claim 7, wherein the shin portion is adjustably secured to the lower knee joint component.

9. A prosthesis according to claim 7, wherein the shin portion is connected to the lower knee joint component proximally relative to the lower pivot so that, at the level of the lower pivot, the shin portion is free to deflect with respect to the lower knee joint component.

10. A prosthesis according to claim 9, wherein the lower knee joint component is in the form of a cradle defining a space which is occupied by the piston and cylinder unit.

11. A prosthesis according to claim 3, wherein said substantially vertically oriented resilient section of the shin portion merges downwardly into a resilient extension which, when the prosthesis is in a static standing position, is posteriorly directed and extends to the ankle portion.

12. A prosthesis according to claim 11, wherein the posteriorly directed section has a cross-section substantially the same as that of the substantially vertically oriented section.

13. A prosthesis according to claim 1, comprising a monolithic fibre-reinforced plastics blade forming the shin portion, the ankle portion, and at least part of the foot portion, the blade being attached to the knee joint.

14. A prosthesis according to claim 1 wherein the vertical distance between the knee axis and the location of the connection of said elongate resilient member to the flexion control device is less than 15 percent of the vertical distance between the knee axis and the lowest weight-bearing surface of the foot portion.

15. A lower limb prosthesis comprising a foot portion, an ankle portion, a resilient shin portion, and a knee joint which defines a knee axis of rotation, wherein the prosthesis further comprises a mounting for attaching the knee joint to a residual limb of an amputee and a control unit interconnecting the mounting and the shin portion, the control unit regulating movement of the shin portion relative to the mounting, wherein the shin portion is attached to the control unit in a region near to the knee axis and extends from its attachment to the control unit to the ankle portion, wherein the knee joint comprises an upper knee joint component and a lower knee joint component which is pivotable relative to the upper knee joint component as the knee joint flexes and which has a shin attachment surface to which the shin portion is secured, the attachment surface being substantially vertical when the prosthesis is in a static standing position, and wherein the shin portion comprises an elongate resilient blade of constant cross-section from its connection to the lower knee joint component and distally thereof, the cross-section having a major axis oriented in a medial-lateral direction.

16. A prosthesis according to claim 15, wherein the shin portion is adjustably secured to the lower knee joint component.

17. A prosthesis according to claim 15, wherein the blade has a first part which is substantially vertical when the prosthesis is in a static standing position and which extends distally from the connection to the lower knee joint component, and a second part into which the first part merges distally, the second part extending distally and posteriorly from the first part to said ankle portion.

18. A prosthesis according to claim 15, wherein the attachment surface is to the anterior of the knee axis.

19. A lower limb prosthesis comprising a mounting portion, a shin portion, an ankle portion, a foot portion, and a knee joint interconnecting the mounting portion and the shin portion and defining a knee axis of rotation, the knee joint further comprising a flexion control device for controlling rotation of the shin portion relative to the mounting portion, the flexion control device including a linear piston and cylinder control device pivotally connected to the mounting portion and the shin portion respectively, the pivotal connections defining upper and lower pivot connection axes, wherein the shin portion comprises an elongate resilient member which is connected to the flexion control device at a location such that, when the prosthesis is in an upright static standing position, the resiliently weight-responsive length of the shin portion extends to a level above the pivot connection axis of the connection between the control device and the shin portion.
